(19) United States
(12) Patent Application Publication
(10) Pub. No.: US 2013/0093995 A1
(43) Pub. Date: Apr. 18, 2013

(54) OPHTHALMIC APPARATUS, OPHTHALMIC IMAGE PROCESSING METHOD, AND RECORDING MEDIUM

(71) Applicants: Nobuhito Suehira, Yokohama-shi (JP); Kazuhiro Matsumoto, Yokohama-shi (JP)

(72) Inventors: Nobuhito Suehira, Yokohama-shi (JP); Kazuhiro Matsumoto, Yokohama-shi (JP)

(73) Assignee: CANON KABUSHIKI KAISHA, Tokyo (JP)

(21) Appl. No.: 13/616,861

(22) Filed: Sep. 14, 2012

(30) Foreign Application Priority Data
Sep. 30, 2011 (JP) 2011-216776

(51) Int. Cl. A61B 3/14

(52) U.S. Cl. CPC A61B 3/14
USPC 351/206; 351/246

(57) ABSTRACT

An ophthalmic apparatus includes a first acquisition unit configured to acquire a first tomogram of a subject's eye, a three-dimensional image acquisition unit configured to acquire a three-dimensional image of the subject's eye after the first tomogram is acquired, a second acquisition unit configured to acquire a second tomogram of the subject's eye corresponding to the first tomogram after the three-dimensional image is acquired, and a correction unit configured to correct a gradation of the second tomogram based on a gradation of the first tomogram.

### BACKGROUND OF THE INVENTION

1. Field of the Invention

The present invention relates to an ophthalmic apparatus and an ophthalmologic image processing method.

2. Description of the Related Art

Various types of ophthalmic equipment using an optical apparatus have been currently used. Examples include an anterior eye imaging machine, a fundus camera, and a confocal scanning laser ophthalmoscope (SLO). Among them, an optical tomography imaging apparatus by optical coherence tomography (OCT) using low coherent light is an apparatus capable of obtaining a tomogram of a subject's eye with high resolution, has been indispensable in an outpatient department specializing in a retina as the ophthalmic equipment, and is hereinafter referred to as an OCT apparatus.

Japanese Patent Application Laid-Open No. 2010-181172 discusses an OCT apparatus equipped with a fundus camera. The fundus camera determines whether an alignment state between a subject's eye and the OCT apparatus and a focus state are appropriate. The fundus camera can determine whether a tomogram preliminarily acquired is appropriate and whether a tracking state of the subject's eye is appropriate. Thus, a measurement can be easily made without missing measurement timing.

In an OCT measurement, an alignment between the OCT apparatus and the subject's eye and focusing are important. However, the tomogram may be unsuccessfully imaged even after such an adjustment is made. The causes include interference with measurement light by an eyelid or an eyelash and movement of eyes. If a 3D measurement is made at a wide angle of view, for example, a tomogram is deteriorated due to the eyelid or the eyelash in the tomogram of an imaging area where an incident position of measurement light is close to the eyelid or the eyelash. Blink and poor fixation may occur during the measurement.

### SUMMARY OF THE INVENTION

Aspects of the present invention are directed to obtaining a good tomogram of a subject's eye even when a factor, which deteriorates an image, occurs in a period of time from an alignment to the end of a measurement.

Besides the above-mentioned object, aspects of the present invention also focus on producing a function and effect that are introduced by each of configurations described in forms for implementing aspects of the invention, described below, and cannot be obtained by the conventional technique as one of other objects according to aspects of the present invention.

According to an aspect of the present invention, an ophthalmic apparatus includes a first acquisition unit configured to acquire a first tomogram of a subject's eye, a three-dimensional image acquisition unit configured to acquire a three-dimensional image of the subject's eye after the first tomogram is acquired, a second acquisition unit configured to acquire a second tomogram of the subject's eye corresponding to the first tomogram after the three-dimensional image is acquired, and a correction unit configured to correct a gradation of the second tomogram based on a gradation of the first tomogram.

According to aspects of the present invention, even when a factor, which deteriorates an image, occurs in a period of time from an alignment to the end of a measurement, a good tomogram of a subject's eye can be obtained.

Further features and aspects of the present invention will become apparent from the following detailed description of exemplary embodiments with reference to the attached drawings.

### BRIEF DESCRIPTION OF THE DRAWINGS

The accompanying drawings, which are incorporated in and constitute a part of the specification, illustrate exemplary embodiments, features, and aspects of the invention and, together with the description, serve to explain the principles of the invention.

FIG. 1 schematically illustrates an example of a configuration of an OCT apparatus according to a first exemplary embodiment.

FIG. 2 schematically illustrates an example of a functional configuration of a computer.

FIG. 3 is a flowchart illustrating signal processing according to the first exemplary embodiment.

FIGS. 4A, 4B, 4C, and 4D respectively illustrate examples of a tomogram during an alignment in the first exemplary embodiment.

FIGS. 5A, 5B, and 5C respectively illustrate examples of a fundus image and a tomogram after a measurement in the first exemplary embodiment.

FIG. 6 schematically illustrates an example of a configuration of an OCT apparatus according to a second exemplary embodiment.

FIG. 7 illustrates an example of a scanning range in the second exemplary embodiment.

FIGS. 8A, 8B, 8C, and 8D respectively illustrate examples of a tomogram during an alignment in the second exemplary embodiment.

### DESCRIPTION OF THE EMBODIMENTS

Various exemplary embodiments, features, and aspects of the invention will be described in detail below with reference to the drawings.

The present invention is not limited to exemplary embodiments, described below, and can be implemented by being modified in various manners without departing from the scope of the present exemplary embodiment.

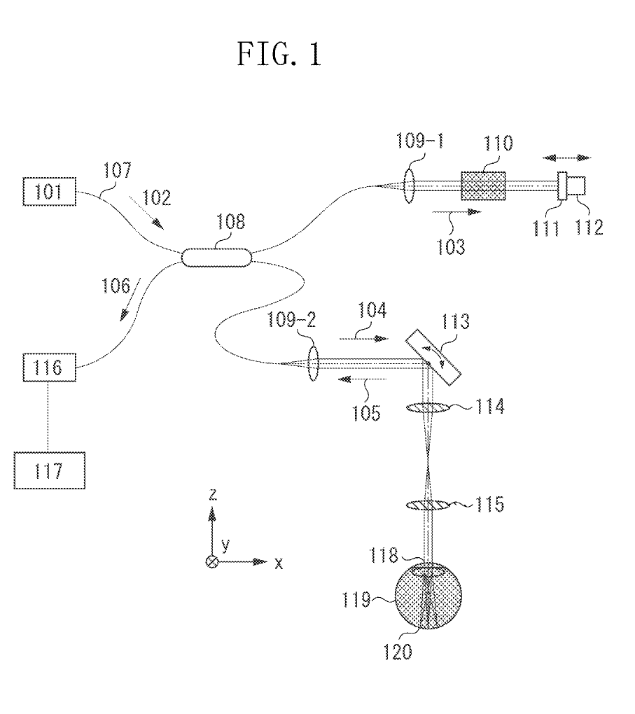

A first exemplary embodiment will be described. FIG. 1 schematically illustrates an example of a configuration of an OCT apparatus according to the first exemplary embodiment. The OCT apparatus includes a Michelson interference system. Output light 102 emitted from a light source 101 is guided by a single mode fiber 107 and is incident on an optical coupler 108, and is split into reference light 103 and measurement light 104 by the optical coupler 108. The measurement light 104 is reflected or scattered by a retina 120 serving as an observation target, to return to the optical coupler 108 as returning light 105. The returning light 105 is combined with the reference light 103, which has passed through a reference light path, by the optical coupler 108, to reach a spectroscope 116 as composite light 106.

The light source 101 is a super luminescent diode (SLD) light source serving as a typical low coherent light source. Near-infrared light is appropriate for a wavelength in view of the fact that eyes are measured. Further, the wavelength may be as short as possible because it affects the resolution in a transverse direction of a tomogram to be obtained. For example, the light source 101 has a center wavelength of 840 nm and a wavelength width of 50 nm. Another wavelength may be selected depending on a measurement site of the observation target. While the SLD light source has been selected as the type of light source, an amplified spontaneous emission (ASE) light source may also be used as long as it can emit low coherent light.

The reference light path for the reference light 103 will be described below. The reference light 103 split by the optical coupler 108 becomes substantially parallel light by a lens 109-1 to be emitted. The reference light 103 then passes through a dispersion compensation glass 110, to change its direction with a mirror 111. The reference light 103 is guided to the spectroscope 116 via the optical coupler 108 again. The dispersion compensation glass 110 compensates the reference light 103 for dispersion occurring when the measurement light 104 travels back and forth to the subject's eye 119 and a scanning optical system. As an example, the average diameter of the eyeball of a Japanese person is estimated to be 24 mm as a typical value. An electric stage 112 can adjust a position of a coherence gate by moving an optical path length of the reference light 103 in a direction indicated by an arrow. The coherence gate means a position at a distance equal to the reference light path in a measurement light path. A computer 117 controls the electric stage 112.

The measurement light path for the measurement light 104 will be described below. The measurement light 104 split by the optical coupler 108 becomes substantially parallel light with a lens 109-2 to be emitted, and is incident on a mirror of an XY scanner 113 constituting the scanning optical system. While the XY scanner 113 has one mirror for simplicity in FIG. 1, the XY scanner 113 actually has two mirrors, i.e., an X scanning mirror and a Y scanning mirror arranged in close proximity to each other. A Z-axis direction is an optical axis direction of the measurement light 104, a direction perpendicular to a Z-axis and horizontal to a sheet surface is an X-axis direction, and a direction perpendicular to the Z-axis and perpendicular to the sheet surface is a Y-axis direction.

The measurement light 104 reaches the subject's eye 119 via a lens 114 and an objective lens 115, to scan the retina 120 with the vicinity of a cornea 118 as a fulcrum. Light, which has been reflected and scattered by the retina 120, returns to a fiber after passing through the objective lens 115, the lens 114, the XY scanner 113, and the lens 109-2. The light is combined with the reference light 103, to reach the spectroscope 116 via the optical coupler 108 as composite light 106.

The composite light 106, which has reached the spectroscope 116, is split for each wavelength by a diffraction grating, and its intensity for the wavelength is detected by a sensor (not illustrated). The computer 117 subjects the composite light 106 to Fourier transformation or the like, to generate a tomogram. The tomogram may optionally be stored in a storage unit in the computer 117 while being displayed on a display unit (not illustrated).

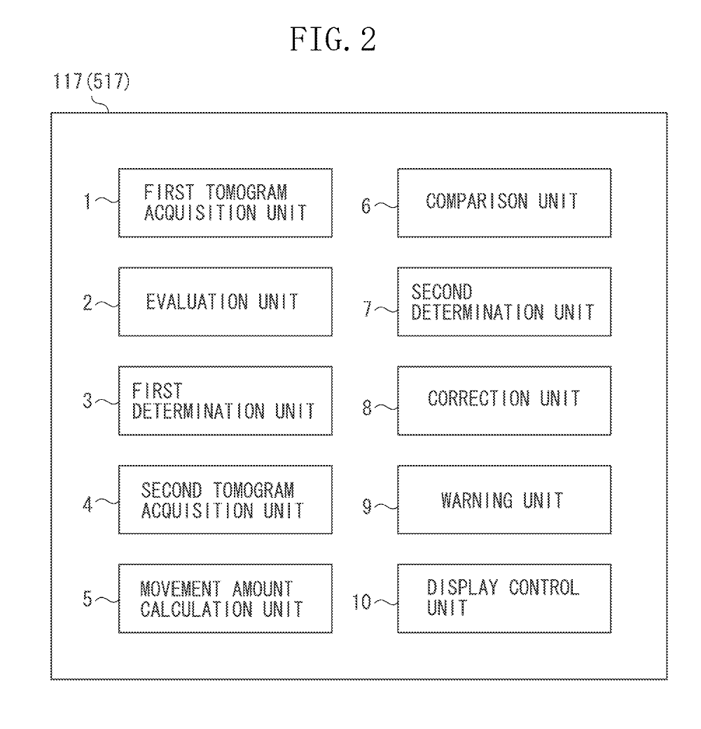

FIG. 2 schematically illustrates an example of a functional configuration of the computer 117.

The computer 117 includes a processing apparatus such as a central processing unit (CPU), and executes a program stored in a storage device such as a memory (not illustrated), to implement various types of functions, described below.

The computer 117 functions as a first tomogram acquisition unit 1, an evaluation unit 2, a first determination unit 3, a second tomogram acquisition unit 4, a movement amount calculation unit 5, a comparison unit 6, a second determination unit 7, a correction unit 8, a warning unit 9, and a display control unit 10.

The first tomogram acquisition unit 1 acquires a tomogram (a first tomogram) of the subject's eye 119 based on an intensity for each wavelength, which has been detected by the sensor, when the ophthalmologic apparatus illustrated in FIG. 1 is aligned with the subject's eye 119. In other words, the first tomogram acquisition unit 1 corresponds to an example of a first acquisition unit that acquires the first tomogram of the subject's eye 119. Specifically, the first tomogram acquisition unit 1 acquires a tomogram in an X-direction by performing scanning in the X-direction with a Y-direction of the XY scanner 113 fixed. The first tomogram acquisition unit 1 acquires a tomogram in the Y-direction by performing scanning in the Y-direction with the X-direction of the XY scanner 113 fixed. The first tomogram acquisition unit 1 alternately continuously performs the above-mentioned processing, to obtain two tomograms, i.e., a tomogram in the X-direction and a tomogram in the Y-direction. The first tomogram acquisition unit 1 may not necessarily acquire the two tomograms, and may acquire only the tomogram in the Y-direction, for example. The first tomogram acquisition unit 1 may acquire a tomogram generated by another computer via wireless or wired based on the intensity for each wavelength that has been detected by the sensor.

The evaluation unit 2 evaluates the tomogram acquired by the first tomogram acquisition unit 1. S, the evaluation unit 2 divides the tomogram acquired by the first tomogram acquisition unit 1 into a plurality of areas, and finds a histogram in each of the areas of the tomogram. For example, the evaluation unit 2 divides the tomogram into three areas, and finds a histogram of the tomogram in each of the areas. The histogram in each of the areas is different from each other among the area including a papilla, the area including a macula, and the area including neither a papilla nor a macula.

The number of areas to be obtained by the division may optionally be changed, and is not limited to three. The evaluation unit 2 is not limited to find a histogram in each of the divided areas of the tomogram but also may find, if the tomogram is divided into three areas, for example, a histogram in the area other than the middle one of a row of the three areas.

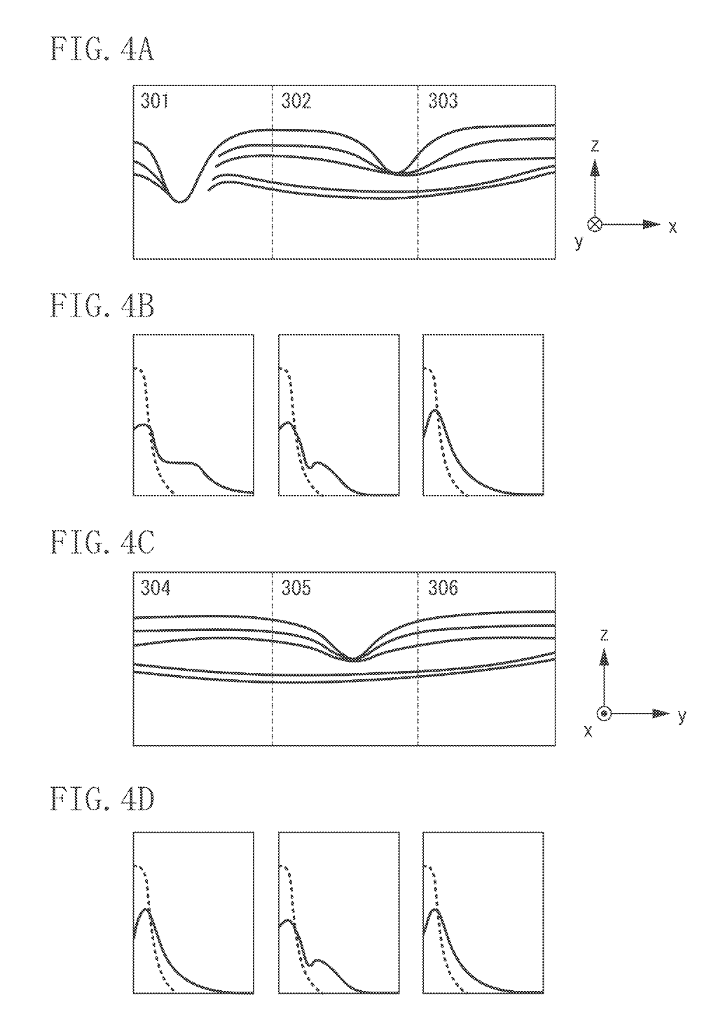

FIG. 4A illustrates an example of a tomogram in the X-direction, and FIG. 4B illustrates an example of a histogram in each of areas 301 to 303 of the tomogram in the X-direction. FIG. 4C illustrates an example of a tomogram in the Y-direction, and FIG. 4D illustrates an example of a histogram in each of areas 304 to 306 of the tomogram in the Y-direction.

The first determination unit 3 determines a state of an alignment (whether an alignment is completed) based on the evaluation by the evaluation unit 2. S, the first determination unit 3 determines the state of the alignment based on the histogram found by the evaluation unit 2. If a left eye is imaged with its macula at its center, for example, the first determination unit 3 subtracts the histogram in the area 303 from the histogram in the area 301 illustrated in FIG. 4A, to determine whether the percentage of cases where a subtraction result is positive in a high-luminance area is a predetermined threshold value or more. Since the area 301 includes an optic papilla, in the histogram in the area 301 the greater frequency on the high-luminance side is greater than the histogram in the area 303. S, the histogram in the area 303 is subtracted from the histogram in the area 301 so that a histogram representing a luminance corresponding to the optic papilla is obtained. In other words, the first determination unit 3 subtracts the histogram in the area 303 from the histogram in the area 301, to determine whether the percentage of cases where a subtraction result is positive in a luminance area corresponding to the optic papilla is the predetermined threshold value or more. While the predetermined threshold value is 80%, for example, it may also be another value.

Even if a right eye is imaged, as is the case with the left eye, an area including no optic papilla is subtracted from an area including an optic papilla, to determine whether the percentage of cases where a subtraction result is positive in a luminance area corresponding to the optic papilla is a predetermined threshold value or more.

Further, the first determination unit 3 performs a subtraction between the respective histograms in the areas 304 and 306 illustrated in FIG. 4C, to determine whether a subtraction result is within a predetermined threshold value, for example. Such a method for determining the alignment uses the fact that a structure of the subject's eye 119 is similar for a straight line passing through a macula and an optic papilla. The predetermined threshold value means, for example, a difference in frequency (the number of pixels) between the area 304 and the area 306 is 5% of pixels in one of the areas. The predetermined threshold value is not limited to this, and may be optionally changed.

The first determination unit 3 determines that the alignment has been successful if the percentage of cases where the subtraction result is positive in the luminance area corresponding to the optic papilla in the tomogram in the X-direction is the predetermined threshold value or more and the difference between the histograms in the areas adjacent to the area including the macula in the tomogram in the Y-direction is less than the predetermined threshold value. In other words the first determination unit 3 determines a state of the alignment based on the first tomogram acquired during the alignment. Specifically, the first determination unit 3 determines a state of the alignment based on a histogram of the first tomogram. More specifically, the first determination unit 3 determines a state of the alignment based on histograms in at least two of a plurality of areas obtained by dividing the first tomogram. The first determination unit 3 determines a state of the alignment based on a difference between histograms of the first tomogram in two areas adjacent to an area including the center of the first tomogram.

The second tomogram acquisition unit 4 acquires a three-dimensional image of the subject's eye 119, and acquires a tomogram (a second tomogram) at a position corresponding to the tomogram acquired by the first tomogram acquisition unit 1 from the three-dimensional image, for example. More specifically, the second tomogram acquisition unit 4 corresponds to an example of a three-dimensional image acquisition unit that acquires the three-dimensional image of the subject's eye 119 after the first tomogram is acquired. Further the second tomogram acquisition unit 4 corresponds to an example of a second acquisition unit that acquires the second tomogram of the subject's eye 119 corresponding to the first tomogram after the three-dimensional image is acquired.

The second tomogram acquisition unit 4 is not limited to acquire the tomogram from the three-dimensional image but may acquire a two-dimensional tomogram constituting the three-dimensional image. The three-dimensional image includes a plurality of tomograms, whether the plurality of tomograms is interpolated or not.

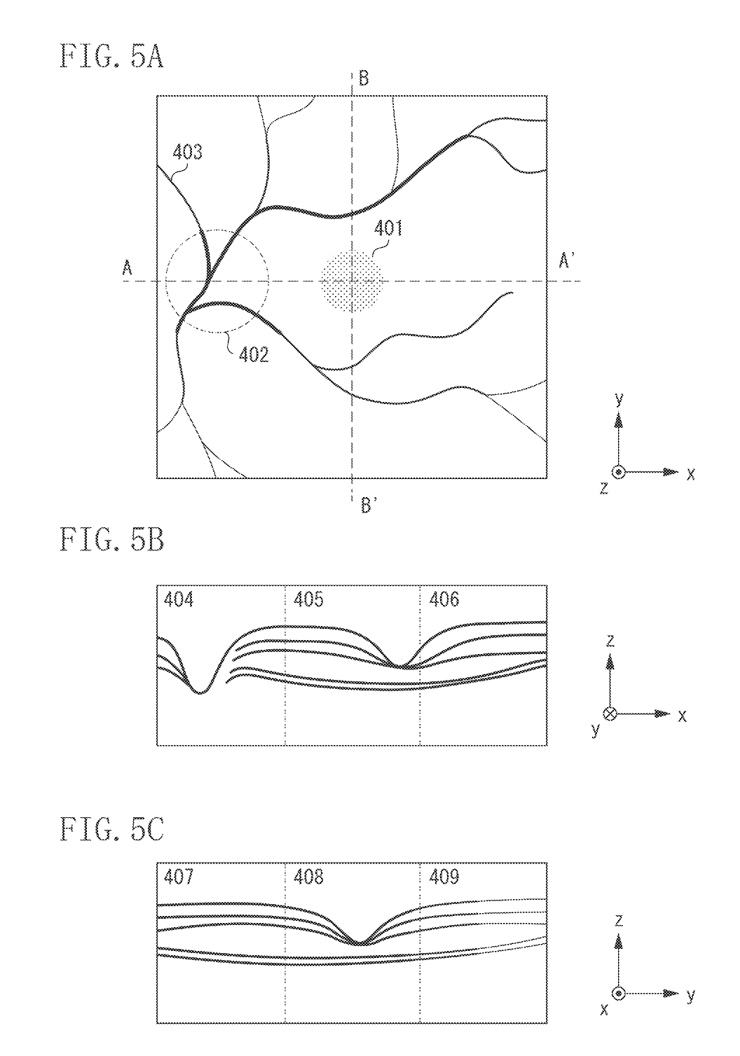

The second tomogram acquisition unit 4 acquires a tomogram in the X-direction and a tomogram in the Y-direction, which correspond to a position where the scanning has been performed during the alignment, from the three-dimensional image, for example. In other words, the second tomogram corresponds to a position of the first tomogram in the subject's eye 119. FIG. 5B illustrates the tomogram in the X-direction, which has been acquired by the second tomogram acquisition unit 4, and FIG. 5C illustrates the tomogram in the Y-direction, which has been acquired by the second tomogram acquisition unit 4. The second tomogram acquisition unit 4 may acquire a tomogram, which has been generated by another computer based on the three-dimensional image, via wireless or wired. Further, the first tomogram acquisition unit 1 may store positional information in the subject's eye 119 of the acquired tomogram, and the second tomogram acquisition unit 4 may acquire a tomogram based on the positional information. If the first tomogram acquisition unit 1 acquires a tomogram with a macula at its center, the second tomogram acquisition unit 4 may acquire a tomogram with a macula at its center after detecting the macula from an fundus image.

The movement amount calculation unit 5 calculates an amount of movement of the subject's eye 119. Specifically, the movement amount calculation unit 5 calculates the amount of movement with reference to FIGS. 4A and 4B and FIGS. 5B and 5C. The amount of movement is calculated by doing a search on which part of FIG. 5B corresponds to a range that matches FIG. 4A.

The movement amount calculation unit 5 determines whether eyes have moved before and after a measurement and how much the eyes have moved during the measurement. The movement of the eyes before and after the measurement is calculated by searching on which part of FIG. 5B corresponds to a range that matches FIG. 4A. The movement amount calculation unit 5 determines whether a tomogram has contracted or expanded in the Y-direction from a magnification of the tomogram because particularly a Y direction is a slow scanning direction for movement in the Z-axis direction during the measurement.

The comparison unit 6 compares the tomogram acquired by the first tomogram acquisition unit 1 and the tomogram acquired by the second tomogram acquisition unit 4. More specifically, the position and the magnification of the tomogram acquired by the second tomogram acquisition unit 4 are corrected based on the amount of movement calculated by the movement amount calculation unit 5, to compare histograms at their corresponding locations.

The second determination unit 7 determines a measurement state of the three-dimensional image of the subject's eye 119 (whether the measurement has been successful) based on a comparison result by the comparison means 6. More specifically, the second determination unit 7 determines that the measurement has been successful if differences in position, magnification, and histogram between the tomogram acquired by the first tomogram acquisition unit 1 and the tomogram acquired by the second tomogram acquisition unit 4 are respectively less than threshold values. If a different portion that has occurred due to the difference in position between the tomogram acquired by the first tomogram acquisition unit 1 and the tomogram acquired by the second tomogram acquisition unit 4 is 10% or less of the entire tomogram, the second determination unit 7 determines that the measurement has been successful. While it is determined that the measurement has been successful when the different portion is 10% or less, the present invention is not limited to this. The value may be changed to various values.

In addition the second determination unit 7 determines that the measurement has been successful if the difference in magnification between the tomogram acquired by the first tomogram acquisition unit 1 and the tomogram acquired by the second tomogram acquisition unit 4 is 2% or less. While it is determined that the measurement has been successful when the difference in magnification is 2% or less, the present invention is not limited to this. The value can be changed to various values.

Further, the second determination unit 7 determines that the measurement has been successful if the difference in histogram between the tomogram acquired by the first tomogram acquisition unit 1 and the tomogram acquired by the second tomogram acquisition unit 4 is 10% or less. The difference in histogram is the ratio of the number of pixels in a different portion between the histogram in each of areas of the tomogram acquired by the first tomogram acquisition unit 1 and the histogram in the area of the tomogram acquired by the second tomogram acquisition unit 4 to the number of pixels in the entire area. While it is determined that the measurement has been successful when the difference in histogram is 10% or less, the present invention is not limited to this. The value may be changed to various values.

The second determination unit 7 acquires a percentage included in a noise level of the histogram in each of the areas of the tomogram if the difference in histogram is 10% or more, to determine whether the tomogram can be corrected. The noise level means previously acquired data obtained when there is no object to be inspected. The second determination unit 7 determines that the tomogram cannot be corrected if the percentage included in the noise level of the histogram in each of the areas of the tomogram is 80% or more. While it is determined that the tomogram cannot be corrected when the percentage is 80% or more, the present invention is not limited to this. The value may be changed to various values.

In other words, the second determination unit 7 determines a measurement state of the three-dimensional image based on the first tomogram and the second tomogram. In other words, the second determination unit 7 determines the measurement state of the three-dimensional image based on the histogram of the first tomogram and the histogram of the second tomogram.

The correction unit 8 corrects the histogram (gradation) of the tomogram acquired by the second tomogram acquisition unit 4 is equal to the histogram (gradation) of the tomogram acquired by the first tomogram acquisition unit 1. More specifically, the correction unit 8 corrects the gradation of the tomogram acquired by the first tomogram acquisition unit 1 so that there is no difference between the histogram of the tomogram acquired by the second tomogram acquisition unit 4 and the histogram of the tomogram acquired by the first tomogram acquisition unit 1. While the correction unit 8 uses γ correction, for example, the present invention is not limited to this. Another method may be used to correct the histogram. Processing for correcting the histogram is performed for the tomogram in the X-direction and the tomogram in the Y-direction. In other words, the correction unit 8 corresponds to an example of a correction unit that corrects the gradation of the second tomogram based on the gradation of the first tomogram. More specifically, the second tomogram acquisition unit 4 corrects the gradation of the second tomogram based on the difference between the histogram of the second tomogram and the histogram of the first tomogram.

Since each of the tomogram in the X-direction and the tomogram in the Y-direction is divided into three areas, as illustrated in FIGS. 5B and 5C in the present exemplary embodiment, the histogram is corrected in three portions of the tomogram. Therefore, a two-dimensional γ distribution, which has been divided into nine portions, is obtained on an XY plane. The correction unit 8 performs histogram correction (γ correction) for the three-dimensional image based on the γ distribution. More specifically, the correction unit 8 corresponds to an example of the correction unit that corrects the gradation of the three-dimensional image based on the difference between the histogram of the first tomogram and the histogram of the second tomogram.

The correction unit 8 corrects the magnification of the tomogram acquired by the second tomogram acquisition unit 4 to be equal to the magnification of the tomogram acquired by the first tomogram acquisition unit 1. In other words, the correction unit 8 corresponds to an example of the correction unit that corrects the magnification of the second tomogram based on the first tomogram. The correction unit 8 adds the noise level data as data that has become insufficient by correcting the magnification, and deletes the data if it has become excessive. Similarly, the correction unit 8 corrects the magnification for the three-dimensional image.

The warning unit 9 issues a warning if the first determination unit 3 determines that the alignment has not been successful. The format of the warning may be a warning by a buzzer sound or display of a display format representing the warning on a display unit by the display control unit 10, described below. The display format representing the warning may be display of characters indicating that the alignment has not been successful, e.g., “confirm alignment” and “during alignment”, or display of a mark, e.g., “x” indicating that the alignment has not been successful.

The warning unit 9 issues a warning if the second determination unit 7 determines that the three-dimensional image has unsuccessfully been measured. The format of the warning may be a warning by a buzzer sound or display of a display format representing the warning by the display control unit 10, described below. The display format representing the warning may be display of characters indicating that the three-dimensional image has not successfully been measured, e.g., “remeasurement is required” and “measurement has been unsuccessful” or display of a mark, e.g., “x” indicating that the three-dimensional image has unsuccessfully been measured. The warning unit 9 may cause the display control unit 10 depending on, for example, the error factor to display “poor fixation”, “light shielding”, and “lack of sensitivity”, respectively, if an error factor is a position or a magnification, a histogram, and a noise level, for example. In other words, the warning unit 9 issues a warning based on a determination result of the state of the alignment by the first determination unit 3. Further, the warning unit 9 issues a warning based on a determination result of the state of the measurement of the three-dimensional image by the second determination unit 7.

The display control unit 10 displays various types of information on the display unit. For example, the display control unit 10 displays a tomogram or a warning which is instructed to display on the display unit from the warning unit 9. In other words, the display control unit 10 displays a display format representing the warning on the display unit based on the determination result of the state of the alignment by the determination unit 3. The display control unit 10 displays a display format representing the warning on the display unit based on the determination result of the measurement state of the three-dimensional image by the second determination unit 7.

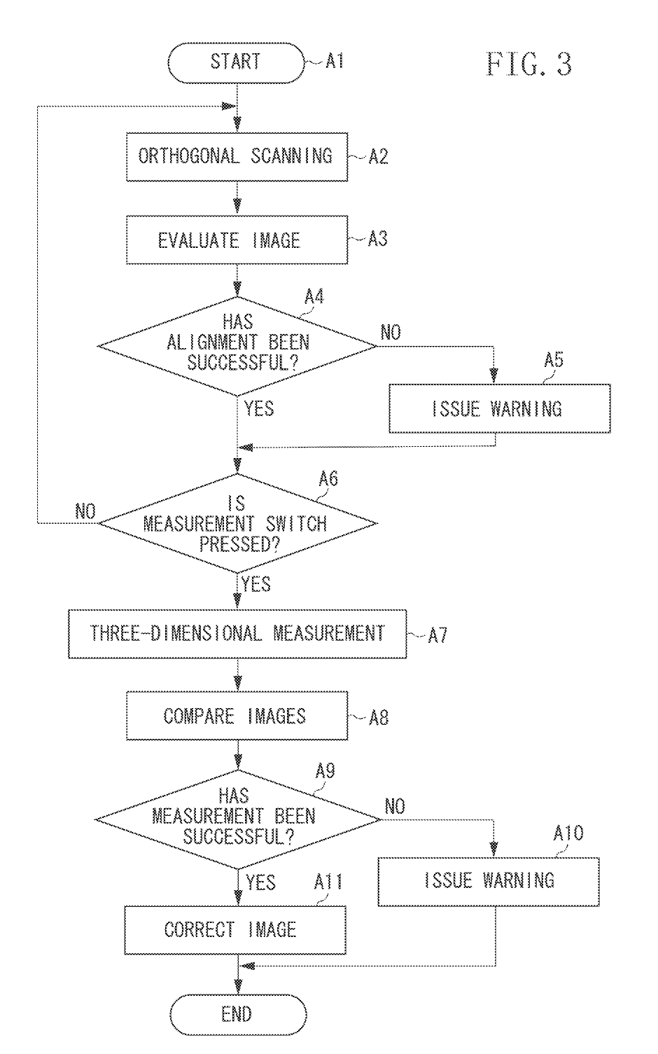

Signal processing for an OCT measurement (an ophthalmic image processing method) will be described with reference to FIG. 3.

In step A1, the measurement is started. In this state, the OCT apparatus has been started, and the subject's eye 119 is arranged at a measurement position.

Steps A2 to A6 are repeated, to align the OCT apparatus and the subject's eye 119 before main imaging. In step A2 (first acquisition step), the first tomogram acquisition unit 1 acquires a tomogram (a prescanned image). Specifically, the first tomogram acquisition unit 1 alternately continuously performs processing for performing scanning in the X-direction with the Y-direction of the XY scanner 113 fixed and performing scanning in the Y-direction with the X-direction thereof fixed, to obtain two tomograms, i.e., a tomogram in the X-direction and a tomogram in the Y-direction. The first tomogram acquisition unit 1 images the tomogram in the X-direction or the Y-direction for each round of a loop of steps A2 to A6. FIGS. 4A and 4C are schematic views of the tomograms acquired in step A2. FIGS. 4A and 4C each illustrate the tomograms in the X-direction and the Y-direction.

FIGS. 4B and 4D each illustrate histograms that will be described in step A3. The tomograms illustrated in FIGS. 4A and 4C are each displayed one above the other, for example, on a part of a screen of the display unit as the tomograms in the X-direction and the Y-direction, respectively. The tomogram is displayed while being sequentially updated for each round of the loop, and is further repeatedly overwritten and stored in a storage unit (not illustrated). For example, the tomogram illustrated in FIG. 4A is imaged immediately before, and the tomogram illustrated in FIG. 4C is imaged before the tomogram illustrated in FIG. 4A is imaged. Data having 1024 lines in the X-direction and 1024 lines in the Y-direction is acquired, assuming that the tomogram is imaged in a width range of 10 mm of a fundus. If the tomogram in the X-direction or the Y-direction finishes being imaged, the processing proceeds to step A3.

In step A3, the evaluation unit 2 evaluates the tomogram acquired in step A2. A histogram is used for the evaluation of the tomogram. Therefore, the evaluation unit 2 finds a histogram of the tomogram. FIG. 4B illustrates histograms in three areas illustrated in FIG. 4A, and the three areas correspond to the areas 301 to 303 from the left respectively. The number of areas is not necessarily three, and may be more than three or less than three. A histogram horizontal axis represents a gray scale (a luminance), and a histogram vertical axis represents a frequency (the number of pixels). A solid line in each of the areas is a histogram in the area. In the histogram in each of the areas, a histogram of noise generated when there is no object is indicated by a dotted line. In other words, when there is no object to be imaged, the pixels have a distribution localized in a low gray scale area. This data has been acquired by imaging the tomogram with nothing installed in a measurement position (in an open state) before the subject's eye 119 is measured, for example. FIG. 4C schematically illustrates a tomogram obtained when scanning has been performed in the Y-direction, and FIG. 4D schematically illustrates histograms in three areas illustrated in FIG. 4C, and the histograms respectively correspond to the histograms in the areas 304 to 306 from the left.

Image evaluation will be described using the histograms illustrated in FIGS. 4B and 4D. The area 301 includes a papilla, and is relatively highly reflective, so that pixels are distributed up to a high gray scale position. The area 302 includes a macula, and the histogram has two bumps, for example. The area 303 is at a position on the opposite side of a papilla across a macula, and is much less highly reflective, so that pixels are distributed from the center of the gray scale to a lower gray scale position. While the areas 304 and 306 are positioned opposite to each other across a macula, so that pixels are distributed in an almost similar manner to those in the area 303 because there is no papilla in both the areas 304 and 306. The area 305 includes a macula, so that pixels are distributed in a similar manner to those in the area 302. If the image evaluation ends, the processing proceeds to step A4.

In step A4, the first determination unit 3 determines whether the alignment has been successful. The first determination unit 3 performs the determination using a previously set threshold value in consideration of measurement sites such as right and left eyes, a papilla, and a macula, the number of divisions of an imaging area based on a measurement mode such as the size of a measurement area, and the type of a site in each of areas obtained by the division. The first determination unit 3 may recognize a layered structure from a tomogram, and compare the layered structure with a previously registered shape, to determine the type of the site included in each of the areas. In this example, the determination is as follows, for example, assuming that the left eye is imaged. The first determination unit 3 subtracts the histogram in the area 303 from the histogram in the area 301, to determine whether there are a large number of cases where a subtraction result is positive in a high-luminance area. Further, the first determination unit 3 performs a subtraction between the respective histograms in the areas 304 and 306, to determine whether cases where a subtraction result is positive and cases where the subtraction result is negative are substantially similar in number and the subtraction result is within a predetermined threshold value. If it is determined that the alignment has been successful (YES in step A4), the processing proceeds to step A6. If it is determined that the alignment has been unsuccessful (NO in step A4), the processing proceeds to step A5.

In step A5, the warning unit 9 issues a warning. If the subtraction result deviates from the threshold value, the display control unit 10 displays a warning such as “confirm alignment” on the display unit. When the warning is displayed, the processing proceeds to step A6. The warning is displayed for a predetermined period of time. The user confirms that the warning is not displayed, to press a measurement switch provided in the computer 117.

In step A6, the computer 117 determines whether the measurement switch (not illustrated) has been pressed. If the measurement switch is pressed (YES in step A6), the processing proceeds to step A7. If the measurement switch is not pressed (NO in step A6), then in step A2, the alignment is performed.

In step A7, the second tomogram acquisition unit 4 performs a three-dimensional measurement (a three-dimensional image acquisition step). For a tomogram having 1024 pixels in the X-direction, data from the spectroscope is acquired at 1024 positions in the Y-direction. Fast scanning and slow scanning are performed in the X-direction and the Y-direction respectively. The data from the spectroscope is stored for each reciprocation in the X-direction. When the spectroscope has 2048 pixels, for example, an array of 1024×2048 pixels is acquired in one reciprocation. When the scanning ends so that all data are stored, a three-dimensional array of 1024×1024×2048 pixels is acquired. Processing is performed for each tomogram (a B-Scan image) acquired by one reciprocation in the X-direction. The tomogram is obtained by subjecting the data from the spectroscope to noise removal, wavelength-wavenumber conversion, Fourier transformation, or the like. As data in a depth direction of the tomogram, for example, 500 pixels are cut out and used. As a result, a three-dimensional array of 1024×1024×500 pixels is obtained as three-dimensional data (a three-dimensional image). FIGS. 5A to 5C illustrate a tomogram in a three-dimensional measurement. FIG. 5A illustrates a two-dimensional image obtained by integrating the data from the spectroscope. The two-dimensional image includes a macula 401, papilla 402, and a vein 403. FIG. 5B illustrates a cross section taken along a line A-A′ in the two-dimensional image, which corresponds to a position where X scanning has been performed during the alignment. FIG. 5C illustrates a cross section taken along a line B-B′ in the two-dimensional image, which corresponds to a position where Y scanning has been performed during the alignment. The second tomogram acquisition unit 4 acquires a tomogram, as illustrated in FIGS. 5B and 5C, from a three-dimensional image (a second acquisition step). When this processing ends, the processing proceeds to step A8.

In step A8, the comparison unit 6 performs image comparison. The comparison unit 6 compares the tomogram acquired in step A7, for example, and the newest tomogram acquired in step A2 immediately before the measurement switch is pressed. The eyes move only within a plane perpendicular to an optical axis during the measurement for simplicity. In other words, in the plane perpendicular to the optical axis direction, an image forming position and a scanning range do not change. When an eyelid or an eyelash blocks light, the tomogram becomes dark. Obviously, if the eyes rotate relative to the optical axis and move in the optical axis direction, 3D data is searched for data closest to data at a position that seems to be measured during the alignment. Thus, the second tomogram acquisition unit 4 acquires data that can be compared with the image during the alignment.

The image comparison is performed with reference to FIGS. 4A and 5B and FIGS. 4C and 5C. The movement amount calculation unit 5 determines whether the eyes move before and after the measurement and how much the eyes move during the movement. An amount of movement of the eyes before and after the measurement is calculated by searching on which part of FIG. 5B corresponds to a range that matches FIG. 4A. An amount of movement in the Z-axis direction of the subject's eye 119 during the measurement is measured from the magnification as to whether the image has contracted and expanded in the Y-direction because the Y-direction is particularly the slow scanning direction. Then, histogram comparison is performed using the histograms illustrated in FIGS. 4A and 5B and the histograms illustrated in FIGS. 4C and 5C. The comparison unit 6 subtracts, from the histograms in the areas 301 to 306, the histograms in the corresponding areas 404 to 409 because it is assumed that the eyes do not move for simplicity. Contrast decreases rightward, i.e., toward the areas 407, 408, and 409 in FIG. 5C. Therefore, a difference occurs between histogram distributions. The comparison unit 6 corrects a position and a magnification, to compare the histograms in corresponding locations when the eyes move. If no parts can be compared in the histograms by the movement, the data is excluded. If the respective numbers of pixels composing the tomogram at during the alignment and the tomogram after the measurement differ, interpolation may optionally be performed, so that the numbers of pixels match each other. If the image comparison ends, the processing proceeds to step A9.

In step A9, the second determination unit 7 determines whether the three-dimensional image has been successfully measured. For example, the second determination unit 7 determines that the measurement has been unsuccessful if differences in position, magnification, and histogram are respectively larger than threshold values. The following are examples of the threshold values. The threshold values are 10% or less, 2% or less, and 10% or less for the position, the magnification, and the histogram respectively. If the difference in histogram is 10% or more, the second determination unit 7 further compares the histogram of the tomogram with a noise level of the tomogram. The noise level of the tomogram is previously acquired data obtained when there is no object to be inspected. Particularly, if an area of the noise level includes 80% of data, the tomogram may be unable to be corrected. If the measurement has been successful (YES in step A9), the processing proceeds to step A11. If the measurement has been unsuccessful (NO in step A9), the processing proceeds to step A10.

In step A10, the warning unit 9 issues a warning. The warning, e.g., “remeasurement is required” is displayed on the display unit. Warnings may be finely classified. “Poor fixation”, “light shielding”, and “lack of sensitivity” may be displayed, if an error factor is a position or a magnification, a histogram, and a noise level respectively. After the warning is displayed, the processing proceeds to step A12.

In step A11 (a correction step), the correction unit 8 performs image correction. The magnification and the histogram may optionally be corrected, even if they are within threshold values for determination. The correction unit 8 adds the noise level data as data that has become insufficient by correcting the magnification, and deletes the data if it has become excessive. The histogram may be corrected using a general method, e.g., γ correction. The γ correction is performed so that the histogram in each of the areas comes closer to the histogram in the area during the alignment. The γ correction is performed on the data in the X-direction and the Y-direction. The γ correction is performed in three portions of each of the tomogram in the X-direction and the tomogram in the Y-direction. However, a two-dimensional γ distribution is obtained for the pixels composing each of the tomograms by using linear interpolation or the like. The correction unit 8 can obtain final three-dimensional data by performing the γ correction in XY coordinates of each of the tomograms based on the two-dimensional γ distribution.

In step A12, the processing ends. Here, a single imaging routine ends. If “remeasurement” is displayed, remeasurement may optionally be performed in step A1 and subsequent steps, by performing another measurement, for example.

As described above, according to the present exemplary embodiment, even when a factor, which deteriorates an image, occurs in a period of time from the alignment to the end of the measurement, a good tomogram of the subject's eye 119 can be obtained.

According to the present exemplary embodiment, the tomogram during the alignment and the tomogram after the measurement are evaluated, so that failure in acquisition of the tomogram due to blink, an eyelash, or movement of eyes can be detected, and appropriate processing such as reperformance of the alignment or reacquisition of the tomogram can be further promoted.

While the two tomograms, which are perpendicular to each other, are acquired and are evaluated in the present exemplary embodiment, processing in which one tomogram, which crosses a main scanning direction during a three-dimensional measurement, has been acquired enables determination of failure in acquisition of the tomogram due to blink, an eyelash, or the like.

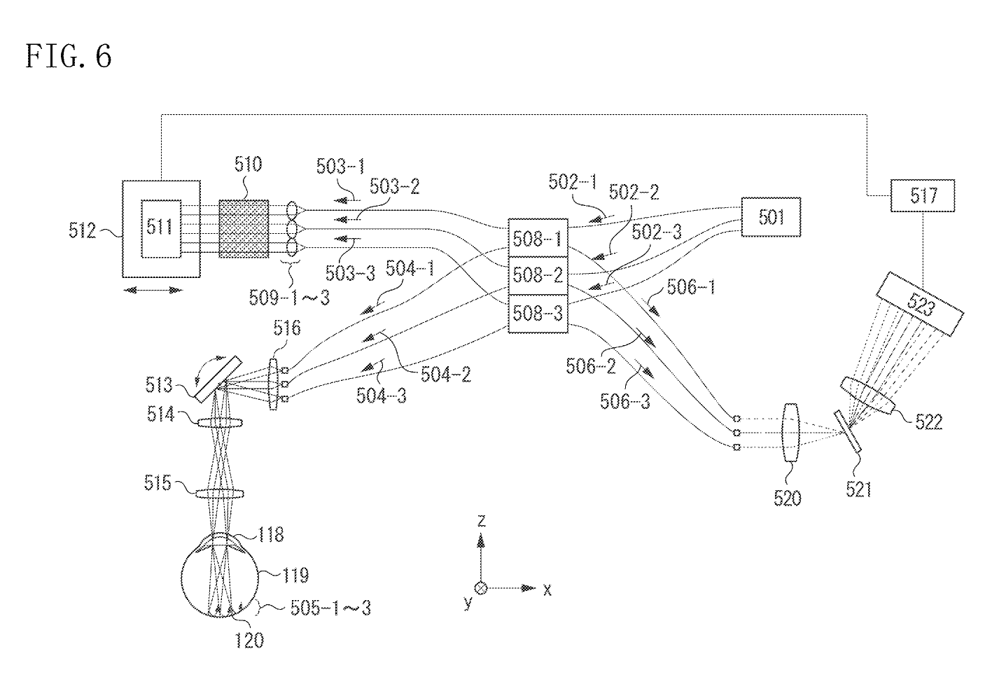

A second exemplary embodiment will be described. FIG. 6 schematically illustrates an example of a configuration of an OCT apparatus according to the second exemplary embodiment.

In the present exemplary embodiment, an example of the OCT apparatus including three measurement light beams will be described. The number of measurement light beams is not limited to this, and may be changed to various values and may be plural number.

Output light emitted from a light source 501 is split into output light beams 502-1 to 502-3 that pass through three light paths, i.e., a first light path, a second light path, and a third light path. Further, each of the three output light beams 502-1 to 502-3 is split into reference light beams 503-1 to 503-3 respectively, and measurement light beams 504-1 to 504-3 respectively, by optical coupler 508-1 to 508-3. Thus-split three measurement light beam 504-1 to 504-3 are respectively reflected or scattered in measurement portions of a retina 120 or the like in a subject's eye 119 serving as an observation target, and are respectively returned as returning light beams 505-1 to 505-3. The returning light beams 505-1 to 505-3 are respectively combined with the reference light beams 503-1 to 503-3 that have passed through a reference light path by the optical couplers 508-1 to 508-3, to become composite light beams 506-1 to 506-3. The composite light beams 506-1 to 506-3 are each divided for each wavelength by a transmissive diffraction grating 521, and are respectively incident on different areas of a line sensor 523. A tomogram of the subject's eye 119 is formed using a signal from the line sensor 523.

The light source 501 is an SLD serving as a typical low coherent light source. One light source is branched into first to third light paths. If an amount of light is insufficient in the one light source, three light sources may be respectively used for the light paths.

The reference light path will be described below. The three reference light beams 503-1 to 503-3 split by the optical couplers 508-1 to 508-3 are respectively changed to substantially parallel light beams with lens 509-1 to 509-3, and are emitted. The reference light beams 503-1 to 503-3 then pass through a dispersion compensation glass 510, change in direction with a mirror 511, and are respectively directed toward the optical couplers 508-1 to 508-3 again. The reference light beams 503-1 to 503-3 respectively pass through the optical couplers 508-1 to 508-3, and are guided to a line sensor 523. The dispersion compensation glass 510 compensates the reference light 503 for dispersion occurring when the measurement light 504 travels back and forth to the subject's eye 119 and a scanning optical system. The average diameter of the eyeball of a Japanese person is estimated to be 24 mm as a typical value. Further, an electric stage 512 can move in a direction indicated by an arrow, and can adjust and control a light path length of the reference light 503. A computer 517 controls the electric stage 512.

A measurement light path of the measurement light 504 will be described below. Each of the measurement light beams 504-1 to 504-3 split by the optical couplers 508-1 to 508-3 is emitted from a fiber end surface, is changed to a substantially parallel light beam with a lens 516, and is incident on a mirror of an XY scanner 513 constituting the scanning optical system. The XY scanner 513 has one mirror for simplicity, however, the XY scanner 513 actually has two mirrors, i.e., an X scanning mirror and a Y scanning mirror arranged in close proximity to each other, and raster-scans the retina 120 in a direction perpendicular to an optical axis. Lenses 514 and 515 are adjusted so that the center of each of the measurement light beams 504-1 to 504-3 substantially matches a rotation center of the mirror serving as the XY scanner 513. The lenses 514 and 515 are optical systems used for the measurement light beams 504-1 to 504-3 to scan the retina 120, and function to scan the retina 120 with the vicinity of a cornea 118 as a fulcrum. Each of the measurement light beams 504-1 to 504-3 is focused at any position on the retina 120.

When incident on the subject's eye 119, the measurement light beams 504-1 to 504-3 are reflected and scattered from the retina 120 to become the returning light beams 505-1 to 505-3, and respectively pass through the optical couplers 508-1 to 508-3, and are guided to the line sensor 523. The foregoing configuration enables the three measurement light beams 504-1 to 504-3 to simultaneously perform scanning.

A configuration of a detection system will be described below. The optical couplers 508-1 to 508-3 respectively combine the returning light beams 505-1 to 505-3 reflected and scattered by the retina 120 and the reference light beams 503-1 to 503-3. Composite light beams 506-1 to 506-3 obtained by the combination are incident on a spectroscope, to respectively obtain spectra. In the spectroscope, the composite light from a fiber is changed to substantially parallel light with a lens 522. The composite light is incident on the transmissive diffraction grating 521 and is dispersed into wavelengths, and is focused on the line sensor 523 with the lens 522. A computer 517 performs signal processing for the spectrum having each of the acquired wavelengths.

FIG. 2 schematically illustrates an example of a functional configuration of the computer 517.

The computer 517 includes a processing apparatus such as a CPU, and executes a program stored in a storage device such as a memory (not illustrated), to implement various types of functions, described below.

The computer 517 functions as a first tomogram acquisition unit 1, an evaluation unit 2, a first determination unit 3, a second tomogram acquisition unit 4, a movement amount calculation unit 5, a comparison unit 6, a second determination unit 7, a correction unit 8, a warning unit 9, and a display control unit 10. Since the functions of the computer 517 are substantially similar to those of the computer 117, detailed description of each of the functions is not repeated.

An example of signal processing for an OCT measurement will be described below with reference to a flowchart illustrated in FIG. 3. A difference from the first exemplary embodiment will be mainly described. Since an operation in the second exemplary embodiment is substantially similar to the operation in the first exemplary embodiment except that a plurality of measurement light beams is used, detailed description of the operation is omitted.

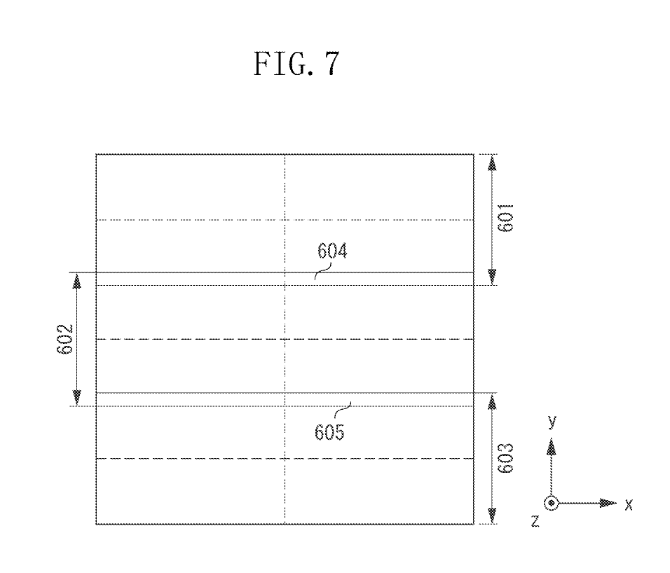

In step A1, the measurement is started. In this state, an OCT apparatus is started, and the subject's eye 119 is arranged at a measurement position. Steps A2 to A6 are repeated, to perform an alignment before main imaging. In step A2, the first tomogram acquisition unit 1 acquires a plurality of tomograms using a plurality of measurement light beams. FIG. 7 illustrates a measurement area by three measurement light beams. Measurement ranges 601 to 603 are respectively covered by the upper, intermediate, and lower measurement light beams. The three measurement light beams are spaced 3.8 mm, for example, apart from one another to perform scanning, to cover a measurement range of 10 mm×10 mm, for example. 20% overlap areas 604 and 605, for example, respectively occur in a scanning range by the upper and intermediate measurement light beams and the intermediate and lower light beams. The three measurement light beams are equally spaced apart from one another in the Y-direction, and move while keeping a positional relationship thereamong in the X-direction and the Y-direction. In other words, the spacing among the three measurement light beams cannot be changed, and the measurement light beams cannot be rotated.

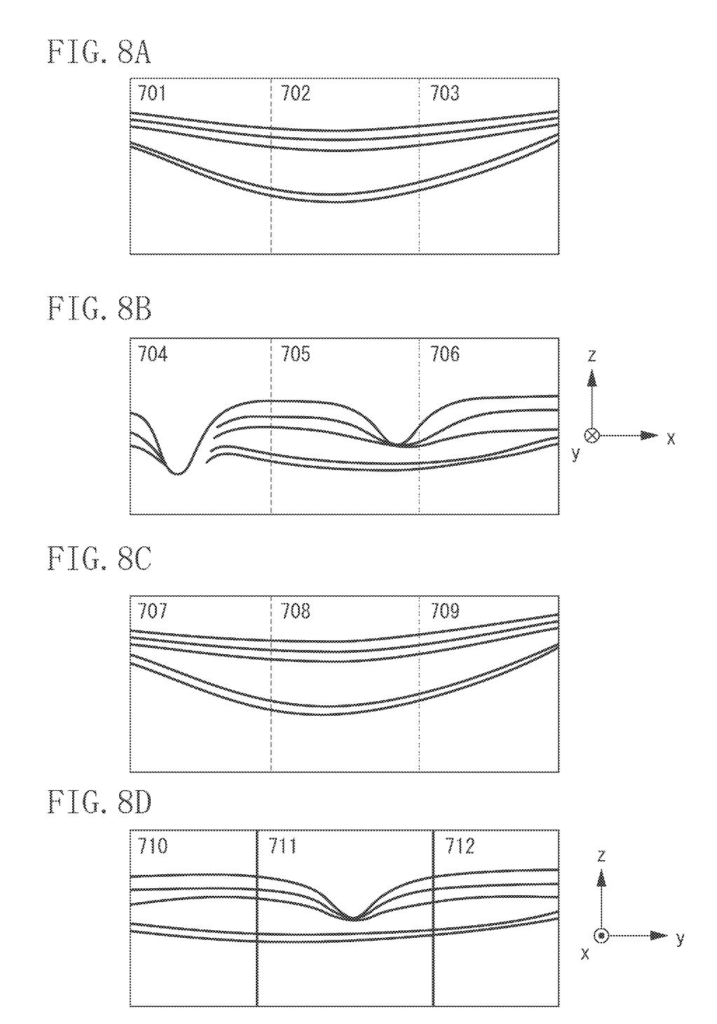

In the alignment, a scanner scans broken-line portions illustrated in FIG. 7 by continuously performing scanning in the X-direction and the Y-direction that are perpendicular to each other. As a result, the first tomogram acquisition unit 1 can simultaneously obtain three tomograms in the X-direction, and can obtain one tomogram by connecting three areas in the Y-direction. The display control unit 10 displays tomograms respectively measured when the scanning is alternately performed in the X-direction and the Y-direction on a screen while recording the tomograms in a storage device. FIGS. 8A to 8D schematically illustrate the tomograms thus acquired. FIG. 8A illustrates the tomogram captured by the upper measurement light beam, FIG. 8B illustrates the tomogram captured by the intermediate measurement light beam, FIG. 8C illustrates the tomogram captured by the lower measurement light beam, and FIG. 8D illustrates the tomogram captured by scanning in the Y-direction. Areas 701 to 712 are obtained by dividing each of the tomograms captured by each of the measurement light beams into three. In the overlap areas, data representing the intermediate measurement light beam, for example, may be used. Respective positions, magnifications, and histograms in the overlap areas are adjusted to be the same previously using a model eye.

In step A3, the evaluation unit 2 evaluates a tomogram. A tomogram acquired by each of the measurement light beams is divided to generate histograms when evaluated. In other words, the evaluation unit 2 divides FIGS. 8A to 8C into respective areas 701 to 709, to generate histograms in the areas 701 to 709. The evaluation unit 2 similarly divides FIG. 8D, to generate histograms in areas 710 to 712.

In step A4, the first determination unit 3 determines whether the alignment has been successful. The first determination unit 3 performs the determination at a previously set threshold value in consideration of measurement modes such as right and left eyes, a papilla, and a macula. As a method for the determination, in this example in which the left eye is captured, the first determination unit 3 subtracts the histogram in the area 703 from the histogram in the area 701 and subtracts the histogram in the area 709 from the histogram in the area 707, to determine whether respective differences therebetween are small (within 5%). The first determination unit 3 subtracts the histogram in the area 706 from the histogram in the area 704, to determine whether the percentage of cases where a subtraction result is positive in a high-luminance area (an area corresponding to the luminance of an optic papilla) exceeds 80%. If the alignment has been successful (YES in step A4), the processing proceeds to step A6. If the alignment has not been successful (NO in step A4), the processing proceeds to step A5.

In step A5, the warning unit 9 issues a warning. A format of the warning is substantially similar to that in the first exemplary embodiment.

In step A6, the computer 517 determines whether a measurement switch (not illustrated) has been pressed. If the measurement switch has been pressed (YES in step A6), the processing proceeds to step A7.

In step A7, the second tomogram acquisition unit 4 performs a three-dimensional measurement. As an example, the second tomogram acquisition unit 4 measures 1024 lines in the X-direction and measures 394 lines in each of areas in the Y-direction, assuming that a range of 10 mm is captured. The second tomogram acquisition unit 4 can also obtain data having 1024 lines in the Y-direction by excluding 79 lines in each of the overlap areas 604 and 605, and can obtain a three-dimensional tomogram by subjecting the acquired data to signal processing.

If an object moves, the second tomogram acquisition unit 4 searches the acquired tomogram for an overlap portion. The second tomogram acquisition unit 4 excludes the overlap portion, to obtain three-dimensional data. At this time, the data may not become the data of 1024 lines in the Y-direction.

The second tomogram acquisition unit 4 acquires a tomogram corresponding to the tomogram acquired by the first tomogram acquisition unit 1 from the three-dimensional image. When this processing ends, the processing proceeds to step A8.

In step A8, the comparison unit 6 performs image comparison. The image comparison is performed by comparing, for each of the tomograms respectively obtained by the measurement light beams, the tomogram during the alignment with the tomogram obtained from the three-dimensional image obtained by a 3D measurement. In other words, the comparison unit 6 compares the tomograms obtained by the upper measurement light beam, the tomograms obtained by the intermediate measurement light beam, and the tomograms obtained by the lower measurement light beam. The comparison unit 6 compares the tomograms in the Y-direction. The movement amount calculation unit 5 calculates how much the eyes move before and after the measurement and how much the eyes move during the measurement before the comparison unit 6 compares the tomograms, like in the first exemplary embodiment. The comparison unit 6 compares the tomograms based on the amount of movement calculated by the movement amount calculation unit 5. For example, the comparison unit 6 finds a difference between the histogram of the tomogram acquired by the first tomogram acquisition unit 1 and the histogram of the tomogram acquired by the second tomogram acquisition unit 4.

In step A9, the second determination unit 7 determines whether the three-dimensional image has been successfully measured. Processing in step A9 is substantially similar to that in the first exemplary embodiment. If the measurement has been successful (YES in step A9), the processing proceeds to step A11. If the measurement has been unsuccessful (NO in step A9), the processing proceeds to step A10.

In step A10, the warning unit 9 issues a warning. A format of the warning is substantially similar to that in the first exemplary embodiment.

In step A11, the correction unit 8 performs image correction. If the histogram is corrected, the correction unit 8 performs correction so that the histogram of the tomogram acquired by the second tomogram acquisition unit 4 comes closer to the histogram of the tomogram during the alignment. If a magnification is corrected, a noise level is inserted into insufficient data, and excessive data is deleted. A two-dimensional, distribution is obtained for pixels composing each of the tomograms, like that in the first exemplary embodiment. The correction unit 8 can obtain final three-dimensional data by performing γ correction in XY coordinates of each of the tomograms based on the γ distribution.

In step A12, the processing ends. While a single measurement ends, the measurement may optionally be performed in step A1 and subsequent steps.

As described above, according to the present exemplary embodiment, in the OTC apparatus using the plurality of measurement light beams, a similar effect to that in the first exemplary embodiment can be obtained.

### Other Embodiments

Aspects of the present invention can also be realized by a computer of a system or apparatus (or devices such as a CPU or MPU) that reads out and executes a program recorded on a memory device to perform the functions of the above-described embodiments, and by a method, the steps of which are performed by a computer of a system or apparatus by, for example, reading out and executing a program recorded on a memory device to perform the functions of the above-described embodiments. For this purpose, the program is provided to the computer for example via a network or from a recording medium of various types serving as the memory device (e.g., a non-transitory computer-readable medium). In such a case, the system or apparatus, and the recording medium where the program is stored, are included as being within the scope of aspects of the present invention.

While the present invention has been described with reference to exemplary embodiments, it is to be understood that the invention is not limited to the disclosed exemplary embodiments. The scope of the following claims is to be accorded the broadest interpretation so as to encompass all modifications, equivalent structures, and functions.

This application claims priority from Japanese Patent Application No. 2011-216776 filed Sep. 30, 2011, which is hereby incorporated by reference herein in its entirety.

1. An ophthalmic apparatus comprising:
a first acquisition unit configured to acquire a first tomogram of a subject's eye;
a three-dimensional image acquisition unit configured to acquire a three-dimensional image of the subject's eye after the first tomogram is acquired;
a second acquisition unit configured to acquire a second tomogram of the subject's eye corresponding to the first tomogram after the three-dimensional image is acquired; and
a correction unit configured to correct a gradation of the second tomogram based on a gradation of the first tomogram.

2. The ophthalmic apparatus according to claim 1, wherein the second acquisition unit acquires the second tomogram from the three-dimensional image.

3. The ophthalmic apparatus according to claim 1, wherein the second tomogram corresponds to a position of the first tomogram in the subject's eye.

4. The ophthalmic apparatus according to claim 1, wherein the correction unit corrects the gradation of the second tomogram based on a difference between a histogram of the second tomogram and a histogram of the first tomogram.

5. The ophthalmic apparatus according to claim 1, wherein the correction unit corrects a gradation of the three-dimensional image based on a difference between a histogram of the first tomogram and a histogram of the second tomogram.

6. The ophthalmic apparatus according to claim 1, wherein the correction unit corrects a magnification of the second tomogram based on the first tomogram.

7. The ophthalmic apparatus according to claim 1, further comprising a first determination unit configured to determine a state of an alignment based on the first tomogram acquired during the alignment.

8. The ophthalmic apparatus according to claim 7, wherein the first determination unit determines the state of the alignment based on the histogram of the first tomogram.

9. The ophthalmic apparatus according to claim 8, wherein the first determination unit determines the state of the alignment based on histograms in at least two or more areas of the first tomogram which is divided into a plurality of areas.

10. The ophthalmic apparatus according to claim 9, wherein the first determination unit determines the state of the alignment based on a difference between histograms of the first tomogram in two areas adjacent to area including the center of the first tomogram.

11. The ophthalmic apparatus according to claim 7, further comprising a second determination unit configured to determine a measurement state of the three-dimensional image based on the first tomogram and the second tomogram.

12. The ophthalmic apparatus according to claim 11, wherein the second determination determines the measurement state of the three-dimensional image based on the histogram of the first tomogram and the histogram of the second tomogram.

13. The ophthalmic apparatus according to claim 7, further comprising a warning unit configured to issue a warning based on a determination result of the state of the alignment by the first determination unit.

14. The ophthalmic apparatus according to claim 13, wherein the warning unit includes a display unit configured to display a display format representing the warning on a display unit based on the determination result of the state of the alignment by the first determination unit.

15. The ophthalmic apparatus according to claim 11, further comprising a warning unit configured to issue a warning based on a determination result of the measurement state of the three-dimensional image by the second determination unit.

16. The ophthalmic apparatus according to claim 15, wherein the warning unit includes a display unit configured to display a display format representing the warning on a display unit based on the determination result of the measurement state of the three-dimensional image by the second determination unit.

17. An ophthalmic apparatus comprising:
a first acquisition unit configured to acquire a first tomogram of a subject's eye;
a three-dimensional image acquisition unit configured to acquire a three-dimensional image of the subject's eye after the first tomogram is acquired;
a second acquisition unit configured to acquire a second tomogram of the subject's eye corresponding to the first tomogram after the three-dimensional image is acquired; and
a correction unit configured to correct a magnification of the second tomogram based on the first tomogram.

18. An ophthalmic image processing method comprising:
acquiring a first tomogram of a subject's eye;
acquiring a three-dimensional image of the subject's eye after acquiring the first tomogram;
acquiring a second tomogram of the subject's eye corresponding to the first tomogram after acquiring the three-dimensional image; and
correcting a gradation of the second tomogram based on a gradation of the first tomogram.

19. A non-transitory recording medium storing a program for causing a computer to execute the method according to claim 18.
